(19) United States
(12) Patent Application Publication
(10) Pub. No.: US 2019/0017038 A1
(43) Pub. Date: Jan. 17, 2019

(54) BEVERAGE WITH HIGH SOLID CONTENT COMPRISING BETA-MANNASE

(71) Applicant: NESTEC S.A., Vevey (CH)

(72) Inventors: Juan Sanz-Valero, Columbus, OH (US); Corietha Cillier, Marysville, OH (US); Rachid Rahmani, Marysville, OH (US); Guillermo Napolitano, Dublin, OH (US); Chitra Kusnadi, Manalapan, NJ (US); Pu-Sheng Cheng, Dublin, OH (US)

(21) Appl. No.: 15/753,223

(22) PCT Filed: Aug. 18, 2016
(86) PCT No.: PCT/EP2016/069564

§ 371 Date: Feb. 16, 2018

Related U.S. Application Data
(60) Provisional application No. 62/208,449, filed on Aug. 21, 2015.

(51) Int. Cl. C12N 9/24; A23F 5/16

(52) U.S. Cl. CPC C12N 9/2494; A23F 5/163; A23F 5/243; A23F 2200/00; C12Y 302/01078

(57) ABSTRACT

The invention relates to compositions for preparing a beverage, the composition comprises a) a coffee liquor or dried coffee extract ranging between 1-40% w/w; b) a creamer ranging between 1-20% w/w; c) a viscosity modifier ranging between 1-15 w/w %; d) an emulsifier ranging between 0.1 to 1%; and e) a β-mannanase ranging between 0.1 to 1% w/w.

### FIELD OF THE INVENTION

The present invention relates to compositions for preparing a beverage with solid contents above 40% (w/w) that is dispersible in cold water (minimum 4° C.) or any other liquid such as milk. The composition comprises a coffee extract, β-mannanase enzyme capable of hydrolysing carbohydrates present in coffee, a creamer, a viscosity modifier comprising sugar-alcohol such as sorbitol; and an emulsifier.

### BACKGROUND OF THE INVENTION

Different mannanase (β-mannanase) preparations are used for the hydrolysis of coffee mannan, thus reducing significantly the viscosity of coffee extracts. Mannan is the main polysaccharide component of these extracts and is responsible for their high viscosity, which negatively affects the technological processing of instant coffee. Mannanase (β-mannanase) may also be used for hydrolyzing poly-galactomannans and poly-glucomannans present in a liquid coffee extract, preferably in order to inhibit gel formation over storage time of the liquid (instant) coffee. In the coffee bean, galactomannans in particular are found. The latter represent approximately 24% of the dry weight of the bean (Bradbury and Halliday, J Agric Food Chem 38, 389-392, 1990). These polysaccharides consist of a linear chain of mannosyl residues which are linked to each other via β-1→4 type linkages and to which are attached α-galactosyl residue monomers. It is also known that the enzyme named endo-β-mannanase (E.C 3.2.1.78) is a hydrolase which degrades (1→4)-β-mannan polymers and releasing small oligosaccharides.

Coffee mannan is isolated from green defatted beans by delignification, acid wash and subsequent alkali extraction with a yield of 12.8% by weight. Additionally, coffee extract polysaccharides are separated by a alcohol precipitation and are found to form nearly half of the coffee extract dry weight. These isolated mannans as well as the mannans in the coffee extract are efficiently hydrolyzed by the mannanase, which resulted in significant viscosity reductions. Concurrently, the reducing sugar content increased continuously due to the release of various mannooligosaccharides including mannotetraose, mannotriose, and mannobiose. Both a partially purified, immobilized and a soluble, crude mannanase preparation are successfully employed for the degradation of coffee mannan.

EP2052078 and EP1086211 relate to modified β-mannanase that has been suggested to be used for many applications in food industry including for coffee extraction and processing of coffee waste.

EP676145 relates to coffee hydrolysis with immobilised β-mannanase.

EP1745702 relate to enzyme-assisted soluble coffee production.

Concentrate beverages such as coffee concentrate products which contain mixtures of soluble coffee solids and other solids such a sugars, flavors, creamers are commonly found in two forms; as soluble beverage powders and as ready-to-drink liquid beverages. Although these products appear to be simple, severe difficulties need to be overcome in order to produce them. In particular, the problem lies in the dispersibility in cold water due to the desirability of high solid content in order to boost coffee taste and creaminess.

The objective of the present invention is to improve the dispersion of the coffee concentrate beverage in cold water.

### SUMMARY OF THE INVENTION

The present invention relates to a shelf-stable beverage concentrate of at least 40% total solids (TS) that easily disperse in cold water (minimum 4° C.) and other liquids such as milk and it is stable over storage time. Furthermore the beverage concentrate is still pourable.

In one aspect, the present invention relates to a shelf-stable liquid beverage comprising at least 40% w/w TS configured to dissolve in cold water comprising:

a) a coffee liquor or dried coffee extract ranging between 1-40% w/w;
b) a creamer ranging between 1-20% w/w
c) a viscosity modifier ranging between 1-15 w/w %;
d) an emulsifier ranging between 0.1 to 1%; and
e) a β-mannanase ranging between 0.1 to 1% w/w.

The surprising aspect of the present invention is to obtain a cold soluble beverage concentrate that contains at least 40% total solids. A person skilled in the art would not be able to produce a cold soluble concentrate that can reach such a high total solid content. FIG. 1A below demonstrates that a composition known in the prior art has issues such as (release from bottle flow rate, undissolved portions, sedimentations, phase separation, clumping).

The above formulation leads to a beverage wherein the viscosity of the continuous aqueous phase of the emulsion in this product is significantly reduced allowing rapid and total dispersion of the concentrated product in cold water (minimum 4° C.).

In yet another aspect, the present invention relates to use of the above beverage concentrate for preparing an instant beverage such as coffee or iced coffee.

In another aspect, the present invention relates to a method of producing a beverage of claim 1 comprising the steps of:

Mixing ingredients as defined in claim 1;

adjusting pH to 5.8 to 7;

Homogenizing the mixture at total pressure ranging from 1400-2500 psi and temperature ranging from 55-75° C.;

Sterilizing at 80-95° C. for 20-100 seconds

Cooling the obtained beverage base product to 25° C. or below; and

Filling in containers.

### BRIEF DESCRIPTION OF THE FIGURES

FIG. 1A shows the main physical defects (sedimentation and phase separation) of the coffee concentrate with 72% total solids (TS) after being reconstituted in cold water. The main ingredients of the product are: coffee, creamer, and sugar. The stirring was done for 10 minutes.

FIG. 1B shows a shelf-stable liquid beverage of present invention dissolved in cold water. The improvement in dispersibility is observed immediately after 10 seconds.

### DETAILED DESCRIPTION OF THE INVENTION

Compositions to be mixed with a coffee extract to prepare a coffee beverage are well known in the art, e.g. milk, cream, coffee whiteners, and coffee creamers. Such compositions are used by consumers to modify e.g. the aroma, appearance and texture of coffee. The compositions may be in liquid or dry form, e.g. as powders, that are dissolved and/or suspended in a cup of coffee, e.g. a cup of freshly brewed coffee or a cup of coffee prepared by dissolving pure soluble coffee in water.

In one embodiment of the invention the composition to be mixed with a coffee extract is a coffee creamer or a coffee whitener. A creamer may e.g. be based on milk protein and/or milk fat, or it may be a non dairy creamer based on vegetable protein and/or vegetable oil or fat. The composition may be in a dry form, e.g. as a powder, wherein the water content is e.g. less than 5%. The composition may also be in a liquid form.

In one embodiment of the invention the total solid content is at least 40% w/w for instance the total solid content typically would range from 40-85% w/w or in range of 60-75% w/w.

In one embodiment of the invention the shelf-stable liquid beverage comprises sugar alcohol as a viscosity modifier. One example of sugar alcohol is soribitol added in concentration ranging from 1-15 w/w %.

The composition of the invention should be formulated such that enzyme will not ferment or react with the composition during storage. This may be achieved e.g. by formulating the composition as a dry powder, and/or by encapsulating the microrganism and/or enzyme so that the microorganisms and/or enzyme will only be released when the composition is mixed with coffee extract or during digestion.

The composition of the invention may further comprise any ingredient suitable for inclusion in a composition to be mixed with a coffee extract to prepare a beverage.

In an embodiment, the product includes addition of sugar, wherein sugar is sucrose up to about 1 to 40 w/w % preferred 20-35.

Usual ingredients may e.g. be sugars, artificial sweeteners, emulsifiers, stabilisers, thickeners, flowing agents, colours, flavours, aromas, and the like. Suitable artificial sweeteners include saccharin, cyclamates, acetosulfame, L-aspartyl based sweeteners such as aspartame, and mixtures of these. Suitable emulsifiers include monoglycerides, diglycerides, lecithin, lactylates, diacetyl tartaric acid esters of mono-diglycerides, emulsifying starches, and mixtures thereof. Suitable stabilisers include dipotassium phosphate and sodium citrate. A suitable flowing agent is sodium silica aluminate. In one embodiment the composition comprises milk protein and/or vegetable protein. In a further embodiment the composition comprises milk fat and/or vegetable oil or fat.

The term “creamer emulsifiers” refer to natural emulsifiers including for example egg yolk. The natural emulsifiers have the advantage of conferring to the finished product improved texture and mouthfeel. In another particular embodiment, the creamer emulsifiers include sugar esters, monoglycerides, diglycerides, esters of monoglycerides and diglycerides, lecithin, lactylates, lysolecithin, polysorbates, sodium stearoyl lactylate and mixtures thereof.

### Coffee

The shelf-stable liquid beverage of the present invention comprises a coffee liquor or dried coffee extract ranging between 1-40% w/w. A coffee extract according to the invention is an extract of green coffee beans and/or roasted coffee beans by water or steam. Numerous methods for producing coffee extracts are known in the art, e.g. from EP 0916267. The coffee extract may e.g. be pure soluble coffee. Pure soluble coffee products are readily available and numerous methods for producing pure soluble coffee products are known in the art, e.g. from EP 106930. The dried coffee extract is freeze-dried or spray-dried.

### Enzymes

A suitable enzyme is a β-mannanase EC 3.2.1.78. The enzyme may be present as a purified enzyme or e.g. in the form of a cell lysate of a microorganism Aspergillus niger. β-mannanase (E.C 3.2.1.78) is a hydrolase which degrades (1-»4) -β-mannan polymers, thus releasing small oligosaccharides. The enzyme should be present in an amount sufficient for hydrolysing carbohydrates present in the coffee extract.

The enzyme may be in any suitable form and added in any suitable way. In one embodiment the enzyme is immobilized, allowing the enzyme to be removed from the composition after treatment and reused. Methods for immobilizing enzymes are well known in the art, and any suitable method may be used.

### Creamers

The shelf-stable liquid beverage of the present invention comprises a creamer that may be diary or non-diary based creamer. In one embodiment, the creamer is non-diary creamer comprising caseinate salts, vegetable oils, creamer emulsifiers and buffer.

The shelf-stable liquid beverage of the present invention may comprise any other ingredient suitable for preparing the desired beverage. Suitable ingredients are well known in the art. Suitable emulsifiers include monoglycerides and derivativesdiglycerides derivatives, lecithin, diacetyl tartaric acid esters of mono-diglycerides (DATEM), emulsifying starches and mixtures thereof. Suitable stabilisers include dipotassium phosphate and sodium citrate. A suitable flowing agent is sodium silica aluminate. In one embodiment the beverage concentrate comprises milk protein and/or vegetable protein. In another embodiment the beverage concentrate comprises milk fat or vegetable fat. In a further embodiment the beverage concentrate comprises a sweetener.

### EXAMPLES

### Example 1

The physical stability and dispersibility in cold water was evaluated in two coffee concentrate products.

Reference sample was produced by mixing water, coffee, creamer, dipotassium phosphate, DATEM, sugar and creamer (containing coconut oil). All ingredients were mixed at 60° C. Once the mixing was completed, the batch was homogenized at 1800 psi and pasteurized. The final total solid content of the product was 72% TS.

Composition of the present invention: It was produced in the same manner with the addition of the following steps: a) Enzymatic treatment of the coffee using a commercially available beta-galactomannanase from Amano (BGM “Amano” 10-K). The coffee (20% w/w) was treated for 30 min at 60 C with an enzyme concentration of 0.5% relative to the amount of coffee. b) Optimization of emulsifier concentration (0.5%) in final recipe to facilitate the dispersion of oil phase in water. c) Addition of 10% w/w sorbitol before adding to the mixture sugar and flavors. The processing conditions and final total solid content was similar to the reference sample.

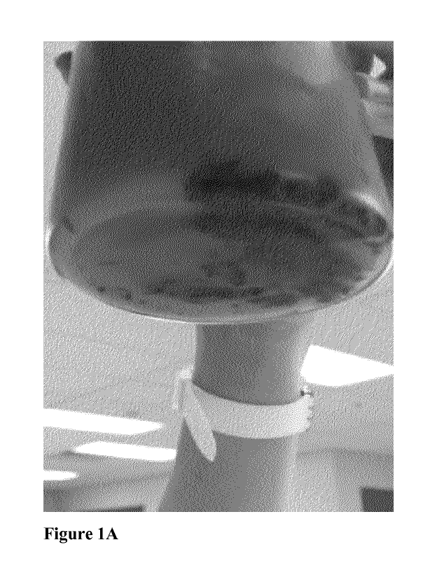

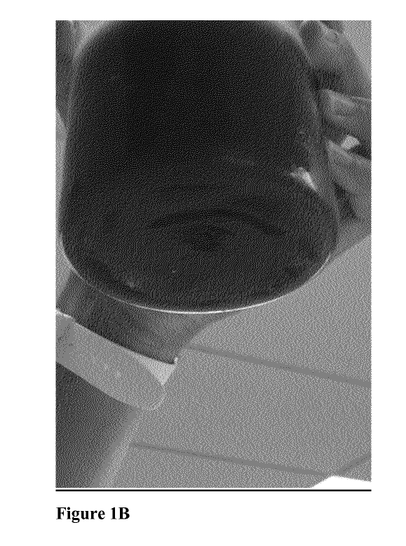

FIG. 1A represents reference sample after reconstitution in cold water (4 C). 10 g of product diluted in 240 g of cold water.

FIG. 1B represents composition of the present invention after reconstitution in cold water (4 C). 10 g of product diluted in 240 g of cold water.

As can be observed in FIG. 1A, reference sample cannot be dispersed properly after 90 seconds of constant agitation. There is a significant amount of sediments in the bottom of the glass. However, composition of the present invention, as shown in FIG. 1B, is completed stable and dispersed in cold water after 10 seconds of constant agitation.

The main difference among these two products was the final viscosity:

| Coffee concentrate products | Viscosity (cp) | TS (%) |
|---|---|---|
| Reference sample | 3180 | 72 |
| Composition of the present invention | 1439 | 72 |

The viscosity of composition of the present invention was reduced in more than 50% respect to reference sample based on all recipe improvements explained above. This drastic reduction in viscosity allowed an easy dispensability in cold water.

The Viscometer Thermo Haake VT500 and SV spindle were used to measure the viscosity at 20 C.

Total Solids were measured by Sand Oven Method

1. A shelf-stable liquid beverage comprising at least 40% w/w total solids configured to dissolve in cold water comprising:
a) a coffee liquor or dried coffee extract of between 1-40% w/w;
b) a creamer of between 1-20% w/w;
c) a viscosity modifier of between 1-15 w/w %;
d) an emulsifier of between 0.1 to 1%; and
e) a β-mannanase of between 0.1 to 1% w/w.

2. The beverage of claim 1 wherein viscosity modifier comprises a sugar alcohol or a polypropylene alcohol.

3. The beverage of claim 2 wherein sugar alcohol is sorbitol.

4. The beverage of claim 1 wherein the dried coffee extract is freeze-dried or spray-dried.

5. The beverage of claim 1 wherein the creamer is diary or non-diary based creamer.

6. The beverage of claim 5 wherein the creamer is non-diary creamer comprising caseinate salts, vegetable oils, creamer emulsifiers and buffer.

7. The beverage of claim 1 wherein emulsifier comprises derivatives of mono and diglycerides, lecithin, lactylates, diacetyl tartaric acid esters of mono-diglycerides, emulsifying starches, and mixtures thereof.

8. The beverage of claim 1 further comprising sugars and/or sweeteners.

9. The beverage of claim 1 wherein β-mannanase is galactomannanase EC 3.2.1.78.

10. A method of producing a beverage comprising the steps of:
mixing ingredients comprising a shelf-stable liquid beverage comprising at least 40% w/w total solids configured to dissolve in cold water comprising: a coffee liquor or dried coffee extract of between 1-40% w/w; a creamer of between 1-20% w/w; a viscosity modifier of between 1-15 w/w %; an emulsifier of between 0.1 to 1%; and a β-mannanase of between 0.1 to 1% w/w;
adjusting the pH to 5.8 to 7;
homogenizing the mixture at total pressure ranging from 1400-2500 psi and temperature ranging from 55-75° C.;
sterilizing at 80-95° C. for 20-100 seconds;
cooling the obtained beverage base product to 35° C. or below; and
filling in containers.

11. A method for enhancing antioxidant capacity in vivo in a human or animal comprising consuming a beverage prepared from a shelf-stable liquid beverage comprising at least 40% w/w total solids configured to dissolve in cold water comprising: a coffee liquor or dried coffee extract of between 1-40% w/w; a creamer of between 1-20% w/w; a viscosity modifier of between 1-15 w/w %; an emulsifier of between 0.1 to 1%; and a β-mannanase of between 0.1 to 1% w/w.
